(19) United States
(12) Patent Application Publication
(10) Pub. No.: US 2025/0143822 A1
(43) Pub. Date: May 8, 2025

(54) ROBOTIC SYSTEM FOR RESPIRATORY DIAGNOSIS AND TREATMENT AND CONTROL METHOD THEREFOR

(71) Applicant: SHENZHEN INSTITUTES OF ADVANCED TECHNOLOGY CHINESE ACADEMY OF SCIENCES, SHENZHEN (CN)

(72) Inventors: WENKE DUAN, SHENZHEN (CN); OLATUNJI MUMINI OMISORE, SHENZHEN (CN); LEI WANG, SHENZHEN (CN)

(21) Appl. No.: 19/014,150

(22) Filed: Jan. 8, 2025

Related U.S. Application Data
(63) Continuation of application No. PCT/CN2022/107882, filed on Jul. 26, 2022.

(30) Foreign Application Priority Data
Jul. 25, 2022 (CN) 202210875746.1

(51) Int. Cl. A61B 34/37; A61B 1/005; A61B 1/267; A61B 5/08; A61B 34/30

(52) U.S. Cl. CPC A61B 34/37; A61B 1/0055; A61B 1/2676; A61B 5/08; A61B 2034/305

(57) ABSTRACT

The present disclosure relates to a robotic system for respiratory diagnosis and treatment and a control method therefor. The present disclosure includes a master control apparatus for operating and controlling a control instruction, a slave control apparatus for receiving the control instruction of the master control apparatus and operating an operation action, and a navigation apparatus for guiding the slave control apparatus. The slave control apparatus includes a slave robot and a slave embedded controller installed inside the slave robot. The slave robot is provided with a mechanical arm, a propulsion support plate connected to the mechanical arm, a biopsy instrument introduction mechanism connected to a propulsion support plate for delivering a biopsy instrument, a bronchoscope rotation driving mechanism for controlling a bronchoscope, and a bending control mechanism for controlling an angle of a flexible end of the bronchoscope.

### CROSS-REFERENCE TO RELATED APPLICATIONS

This application is a continuation of co-pending International Patent Application Number PCT/CN2022/107882, filed on Jul. 26, 2022, the disclosure of which is incorporated herein by reference in its entirety.

### TECHNICAL FIELD

The present disclosure relates to the technical field of medical machinery, and in particular to a robotic system for respiratory diagnosis and treatment and a control method therefor.

### BACKGROUND

According to the International Cancer Research Institute of the World Health Organization, a number of cancer victims in the world is rapidly increasing. Lung cancer is the largest death rate in the year. In 2020, about 1.8 million people died of lung cancer. Early detection is the key to the diagnosis and treatment of lung cancer, and the benign and malignant of small pulmonary nodules is the important basis for early detection of lung cancer. Clinical practice has demonstrated that early diagnosis and early intervention of the small pulmonary nodules have a very significant prognostic effect, even allowing many patients with early lung cancer to be completely eradicated, significantly reducing mortality rate.

Bronchoscopy biopsy and percutaneous biopsy are two common biopsy methods. Bronchoscopy biopsy determines a location of pulmonary lesions by CT or X-ray scan. A diagnosis rate of central lesions at the hilum of the lung is high, and the injury to the human body is less than percutaneous biopsy. When doctors are close to patients with respiratory infections such as COVID-19, severe acute respiratory syndrome, and Middle East respiratory syndrome (MERS), there is a certain risk of infection. And then a biopsy operation is performed, a high concentration virus in lower respiratory tract mucus is released to form a sol during the operation, which causes a higher risk of infection for medical workers. Even if the medical workers use protective measures such as wearing protective suit, medical protection mask, and protection screen, a mental obstacle of a virus threat is inevitable, so that a diagnosis time is prolonged, and a doctor-patient risk is increased. In addition, multiple factors such as long-term X-ray radiation, physician fatigue, and hand operation instability, etc., during an interventional biopsy operation also negatively affect a quality of the operation, and increase a risk of the operation. Using a robot technology can minimize the foregoing risk. Meanwhile, an internal bronchial environment is complex, and a doctor needs to operate multiple devices and operations at the same time.

### SUMMARY

The present disclosure provides a robotic system for respiratory diagnosis and treatment, the system sends a control instruction by a master control apparatus, receives the control instruction of the master control apparatus to perform an operation action by a slave control apparatus, and tracks and guides the slave control apparatus to deliver a biopsy instrument and clamp lesion tissue by a navigation apparatus. The present disclosure also provides a robotic control method for respiratory diagnosis and treatment.

To resolve the foregoing problem, a technical solution used in the present disclosure is to provide a robotic system for respiratory diagnosis and treatment, comprising:
a master control apparatus, configured to perform and control to send a control instruction;
a slave control apparatus, configured to receive the control instruction of the master control apparatus and performing an operation action; and
a navigation apparatus, configured to track and guide the slave control apparatus; and
wherein the slave control apparatus comprises a slave robot and a slave embedded controller installed in the slave robot, and the slave robot comprises a mechanical arm positioned on the slave robot, a propulsion support plate connected to the mechanical arm, a biopsy instrument introduction mechanism connected to the propulsion support plate and configured to deliver a biopsy instrument, and a bronchoscope rotation driving mechanism configured to control a bronchoscope.

In an improvement of the present disclosure, the slave robot further comprises a bending control mechanism connected to the bronchoscope rotation driving mechanism and configured to adjust an end catheter of the bronchoscope.

In an improvement of the present disclosure, the bronchoscope rotation driving mechanism comprises a rotation mechanism motor, a worm, a bearing support, a worm wheel, and a rotation support shaft seat, the rotation mechanism motor is connected to the propulsion support plate by a motor support, the bearing support is fixedly connected to the propulsion support plate, the rotation mechanism motor is connected to the bearing support by the worm, the worm engages with the worm wheel, the rotation support shaft seat is connected to the worm wheel, the rotation support shaft seat is connected to the propulsion support plate by a rotation support shaft.

In an improvement of the present disclosure, the bending control mechanism comprises a bending control knob, a bending control knob groove, a worm wheel rotation sliding block, a bending control worm, and a bending control motor, and the bending control motor and the worm wheel rotation sliding block are connected to the propulsion support plate, the bending control motor is connected to the bending control worm, the bending control worm engages with a worm wheel positioned inside the worm wheel rotation sliding block, and the bending control knob is received in the bending control knob groove defined in the worm wheel rotation sliding block and configured to lock the rotation support shaft.

In an improvement of the present disclosure, the biopsy instrument introduction mechanism comprises an introduction motor, a conveying roller, an inert wheel fixing shaft, an inert wheel, a guide rail, a spring, and a guide rail stopper, and the introduction motor is connected to a base plate; and the base plate is connected to the propulsion support plate, the conveying roller is connected to the introduction motor, the inert wheel is axially connected to the inert wheel fixing shaft, the inert wheel fixing shaft is connected to the guide rail and is connected to the guide rail stopper by the spring, and the inert wheel and the conveying roller are configured to guide the biopsy instrument to be rolled into a biopsy channel of the end catheter of the bronchoscope.

In an improvement of the present disclosure, the master control apparatus comprises a master computer host electrically connected to a master robot, and the master computer host comprises a motion controller configured to send the control instruction.

In an improvement of the present disclosure, the navigation apparatus comprises a visual display mechanism configured to display and an electromagnetic navigation end mechanism configured to guide the slave control apparatus.

In an improvement of the present disclosure, the visual display mechanism comprises a visual flexible laryngoscope.

In an improvement of the present disclosure, the electromagnetic navigation end mechanism comprises an electromagnetic sensor, the electromagnetic navigation system is installed on a tray on an operating table, and the electromagnetic navigation system comprises a magnetic field generator.

A robotic control method for a respiratory diagnosis and treatment, comprising the following steps:
step S1, fixing a bronchoscope to a slave robot of a slave control apparatus;
step S2, operating a master control apparatus to send a control instruction to control the slave robot to deliver a bronchoscope and a biopsy instrument to a predetermined aspiration and biopsy position in a respiratory tract of a patient;
step S3, controlling the biopsy instrument to perform an operation, and take out a lesion tissue;
step S4, putting the taken-out lesion tissue on a slide, fixing the lesion with alcohol, and immediately sending the lesion tissue to a pathological examination;
step S5, controlling the slave robot to withdraw the bronchoscope and the biopsy instrument from the respiratory tract of the patient;
step S6, disinfecting the slave robot.

Beneficial effects of the present disclose is as follows: compared with the prior art, in the present disclosure, the master control apparatus sends a control instruction, and the slave control apparatus receives the control instruction of the master control apparatus to perform an operation action, and the navigation apparatus tracks and guides the slave control apparatus to deliver a biopsy instrument and clamp lesion tissue.

### BRIEF DESCRIPTION OF DRAWINGS

FIG. 1 is a block diagram of a robotic system for respiratory diagnosis and treatment according to an embodiment of the present disclosure.

FIG. 2 is a schematic diagram of controlling of the robotic system for respiratory diagnosis and treatment, according to an embodiment of the present disclosure.

FIG. 3 is a schematic diagram of diagnosis and treatment of a slave robot according to, an embodiment of the present disclosure.

FIG. 4 is a schematic diagram of a propulsion part of a slave robot, according to an embodiment of the present disclosure.

FIG. 5 is a schematic diagram of a bronchoscope rotation driving mechanism of the slave robot, according to an embodiment of the present disclosure.

FIG. 6 is a schematic diagram of a flexible end bending control mechanism of a bronchoscope of the slave robot, according to an embodiment of the present disclosure.

FIG. 7 is a schematic diagram of a biopsy instrument introduction mechanism of the slave robot, according to an embodiment of the present disclosure.

FIG. 8 is a schematic diagram of a telescopic catheter of the slave robot, according to an embodiment of the present disclosure.

FIG. 9 is a schematic diagram of an electromagnetic navigation end mechanism of the slave robot, according to an embodiment of the present disclosure.

### DESCRIPTION OF EMBODIMENTS

To make the objectives, technical solutions, and advantages of the present disclosure clearer and more comprehensible, the following further describes the present disclosure in detail with reference to the accompanying drawings and embodiments. It should be understood that the specific embodiments described herein are merely intended to explain the present disclosure, but are not intended to limit the present disclosure.

At present, the Monarch robot of the Auris Health company and the Ion robot of the Intuitive Surgery company drive an endoscope and a biopsy needle by two mechanical arms and two rotating wheels on the mechanical arms. To adapt to the rotating wheels, the endoscope and the biopsy needle of the Monarch robot both need to be specially designed, which causes a great burden on medical costs.

In addition, the Ion robot is a new robot platform of the Intuitive company, and configured for a minimally invasive biopsy around the lungs. This system uses an ultra-thin hinged robot catheter. An intraoperative navigation manner uses an optical fiber shape sensing technology. An optical fiber needs to pass through the entire endoscope. After navigation, an endoscope lens must be removed from the catheter. A working channel needs to accommodate sampling tools. Thus, the Ion robot cannot visualize a bronchial internal environment in real time during biopsy sampling, which poses a great threat to the surgical safety.

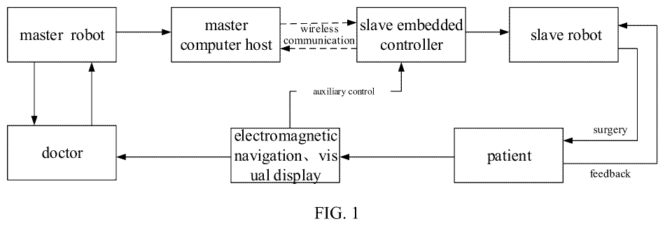

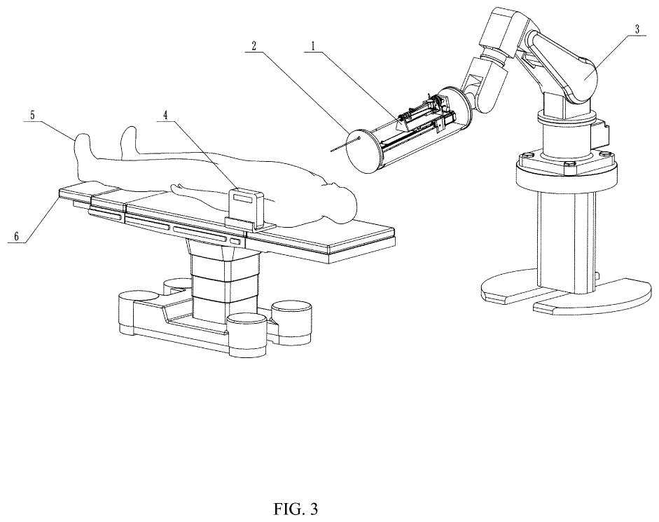

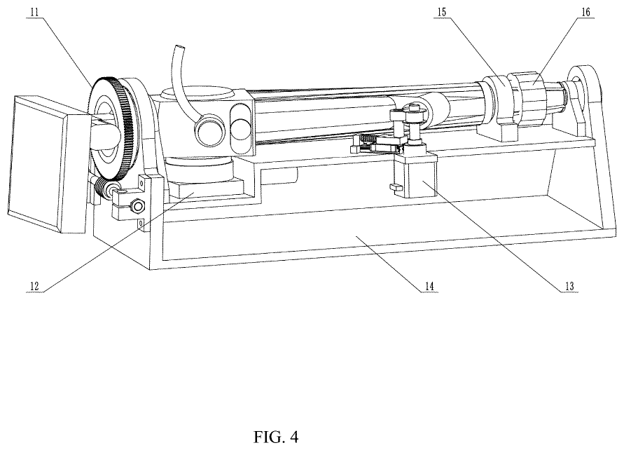

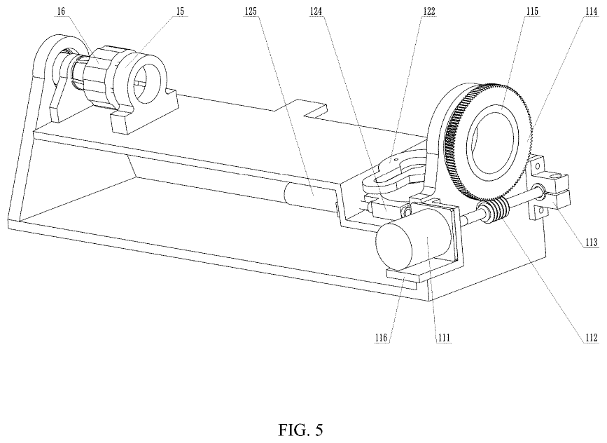

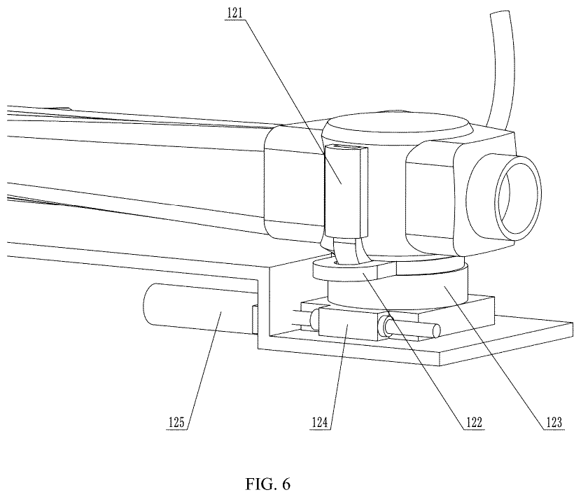

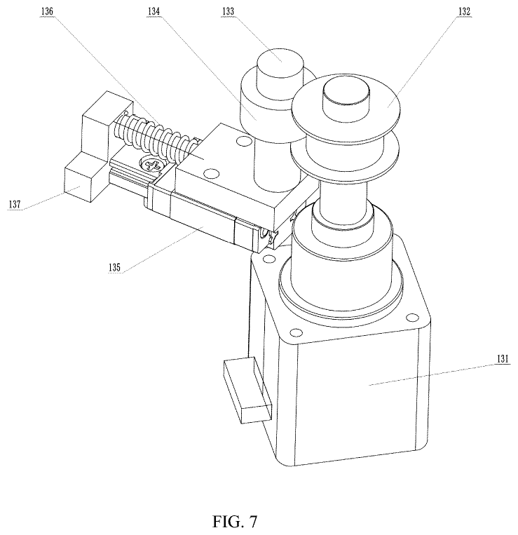

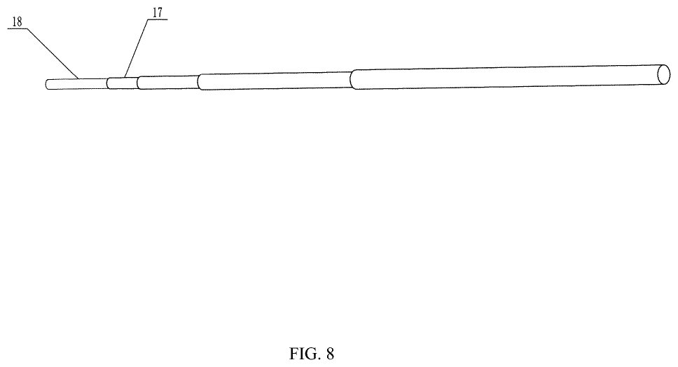

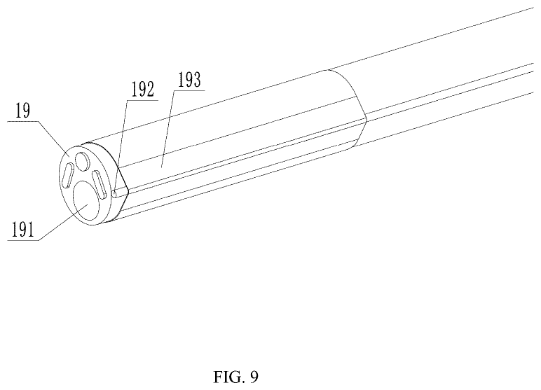

Referring to FIG. 1 to FIG. 9, a robotic system for respiratory diagnosis and treatment according to the present disclosure includes:
a master control apparatus, configured to perform and control to send a control instruction;
a slave control apparatus, configured to receive the control instruction of the master control apparatus to perform an operation action; and
a navigation apparatus, configured to track and guide the slave control apparatus; and
wherein, the slave control apparatus includes a slave robot and a slave embedded controller installed in the slave robot. The slave robot includes a mechanical arm 3, a propulsion support plate 14 connected to the mechanical arm, a biopsy instrument introduction mechanism 13 connected to the propulsion support plate 14 and configured to deliver a biopsy instrument, and a bronchoscope rotation driving mechanism 11 configured to control a bronchoscope.

The present disclosure sends the control instruction by the master control apparatus, receives the control instruction of the master control apparatus to perform the operation action by the slave control apparatus, and meanwhile guides the slave control apparatus by the navigation apparatus tracks, to deliver the biopsy instrument and clamp lesion tissue.

In this disclosure, a doctor operates the master control apparatus of an operating robot, detects instruction information of the master control apparatus in real time, sends the control instruction to the slave control apparatus by a wireless network. The slave control apparatus operates a flexible bronchoscope to perform corresponding propulsion, retraction, rotation, and tip bending control actions. Meanwhile, the slave robot cooperatively uses an electromagnetic navigation technology and a visual navigation function provided by the bronchoscope to accurately locate a position of the bronchoscope in real time.

In this disclosure, the master control apparatus includes a master computer host and a master robot electrically connected to each other. The master computer host includes a motion controller configured to send the control instruction. The master robot uses a common force feedback device. The master computer host is connected to the host robot by an IEEE 1394 interface. The motion controller and the master computer host receive an operation command of the master robot by a network adapter, and process the operation command to call and send an angle library motion instruction the slave control apparatus.

As shown in FIG. 1, the robotic system for respiratory diagnosis and treatment in present disclosure uses a computer host as a development platform and uses a control manner of an upper computer and a lower computer.

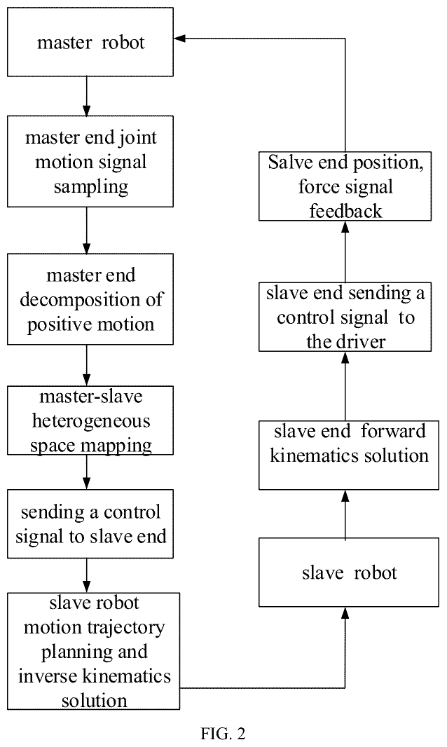

As shown in FIG. 2, the robotic system for respiratory diagnosis and treatment in present disclosure includes a master robot, a control system connected to the master robot, a motion database, a slave robot, and a slave embedded controller. The slave robot receives an instruction sent by the slave embedded controller, thereby completing an operation of a corresponding task object by a driver, for example, pushing or pulling, rotating, and a bending control. Meanwhile, the slave robot feeds back location and speed information to the slave embedded controller by the driver.

The disclosure can further include a signal conversion unit. The signal conversion unit is configured to set an initial value of an encoder, and the signal conversion unit is connected to an industrial PC by an RS485 interface. The signal conversion unit is connected to a driver by the RS422 interface. The upper computer of the control system connects the master robot, the motion controller, a data collection card, etc. by a peripheral hardware interface and a bus. The upper computer of the control system transmits an operation instruction of the master robot to the master computer. The master computer receives a motion state of the robot, processes the operation command by using a control algorithm, and generates and sends a motion instruction to a motion control card by an Ethernet card. The motion control card that controls the lower computer of the control system receives the control command of the master computer, and drives a DC motor to enable the robot to complete a corresponding action. The lower computer sends a joint encoded value from the current slave robot to the master computer.

This disclosure provides an embodiment. This embodiment includes:
a master control apparatus, configured to perform and control to send a control instruction;
a slave control apparatus, configured to receive the control instruction of the master control apparatus to perform an operation; and
a navigation apparatus, configured to track and guide the slave control apparatus; and
the slave control apparatus includes a slave robot and a slave embedded controller installed in the slave robot. The slave robot includes a mechanical arm 3, a propulsion support plate 14 connected to the mechanical arm, a biopsy instrument introduction mechanism 13 connected to the propulsion support plate 14 and configured to deliver a biopsy instrument, a bronchoscope rotation driving mechanism 11 configured to control a bronchoscope, and a bending control mechanism 12 connected to the bronchoscope rotation driving mechanism 11 and configured to adjust an end catheter of the bronchoscope.

In this embodiment, the bronchoscope rotation driving mechanism 11 includes a rotation mechanism motor 111, a worm 112, a bearing support 113, a worm wheel 114, and a rotation support shaft seat 115. The rotation mechanism motor 111 is connected to the propulsion support plate 14 by a motor support 116. The bearing support 113 is fixedly connected to the propulsion support plate 14. The rotation mechanism motor 111 is connected to the bearing support 113 by the worm 112. The worm 112 engages with the worm wheel 114. The rotation support shaft seat 115 is connected to the worm wheel 114. The rotation support shaft seat 115 is connected to the propulsion support plate 14 by the rotation support shaft.

In this embodiment, the bending control mechanism 12 includes a bending control knob 121, a bending control knob groove 122, a worm wheel rotation sliding block 123, a bending control worm 124, and a bending control motor 125. The bending control motor 125 and the worm wheel rotation sliding block 123 are connected to the propulsion support plate 14, the bending control motor 125 is connected to the bending control worm 124. The bending control worm 124 engages with a worm wheel disposed in the worm wheel rotation sliding block 123. The bending control knob 121 received in the bending control knob groove 122 defined in the worm wheel rotation sliding block 123, and configured to lock the rotation support shaft.

In this embodiment, the biopsy instrument introduction mechanism 13 includes an introduction motor 131, a conveying roller 132, an inert wheel fixing shaft 133, an inert wheel 134, a guide rail 135, a spring 136, and a guide rail stopper 137. The introduction motor 131 is connected to a base plate. The base plate is connected to the propulsion support plate 14. The conveying roller 132 is connected to the introduction motor 131. The inert wheel 134 is axially connected to the inert wheel fixing shaft 133. The inert wheel fixing shaft 133 is connected to the guide rail 135 and is connected to the guide rail stopper 137 by the spring 136. The inert wheel 134 and the conveying roller 132 are configured to guide the biopsy instrument to be rolled into a biopsy channel at the end catheter of the bronchoscope.

In this embodiment, the navigation apparatus includes a visual display mechanism configured to display and an electromagnetic navigation end mechanism 19 configured to guide the slave control apparatus. The visual display mechanism includes a visual flexible laryngoscope. The electromagnetic navigation end mechanism 19 includes an electromagnetic sensor. An electromagnetic navigation system is installed on a tray on an operating table 6. The electromagnetic navigation system includes a magnetic field generator. Specifically, the electromagnetic navigation system is placed on the tray fixed to the operating table 6 by bolts, and is cooperatively applied with the electromagnetic navigation end mechanism 19 by an electromagnetic signal. The electromagnetic navigation end mechanism 19 includes a biopsy channel 191, an electromagnetic sensor 192, and a sensor fixing ring 193. The sensor fixing ring 193 secures the electromagnetic sensor 192 by sticking. The electromagnetic sensor 192 senses location and shape information of a bronchoscope catheter 18 in real time, and guides the biopsy instrument to accurately reach a lesion by the biopsy channel 191.

In this embodiment, a slave robot body 1 connects to the propulsion support plate 14 to a mobile slide table by bolts, to complete a propulsion process of the bronchoscope. A lens body fixing nut 16 is connected to a lens body fastener by thread to secure the bronchoscope. A telescopic rod 17 is fixed to the end rotating support shaft seat 115. There are four 150 mm telescopic rod nested connection to guide the end catheter of the bronchoscope to be introduced from a proximal end.

This disclosure provides a robot control method for controlling respiratory diagnosis and treatment, including the following steps:

Step S1, fixing a bronchoscope to a slave robot of a slave control apparatus.

Step S2, operating the master control apparatus to send a control instruction to control the slave robot to deliver the bronchoscope and the biopsy instrument to a predetermined aspiration and biopsy position in a respiratory tract of a patient.

Step S3, controlling the biopsy instrument to perform an operation, and take out a lesion tissue.

Step S4, putting the taken-out lesion tissue on a slide, fixing the lesion tissue with alcohol, and immediately sending the lesion tissue to a pathological examination.

Step S5, controlling a slave robot to withdraw the bronchoscope and the biopsy instrument from the respiratory tract of the patient.

Step S6, disinfecting the slave robot.

Specifically, the workflow includes:

1. Installing and debugging the robot to fix with a bronchoscope. Installing the disinfected slave robot on the mechanical arm, and fix the bronchoscope on the slave robot.

2. The doctor uses the master robot to control the slave robot, and delivers the bronchoscope to a predetermined aspiration and biopsy position in a respiratory tract of a patient.

3. If a biopsy forceps is used for forceps testing, the doctor manually delivers the biopsy forceps to a head portion of the bronchoscope. The biopsy instrument introduction mechanism sends the biopsy forceps by the friction wheel to reach into the lesion and clamp the lesion. If a biopsy needle is used for the needle aspiration biopsy, the biopsy needle is sent by the bronchoscope. The biopsy instrument is delivered by the friction wheel. After the bronchoscope is exposed at a front end of the needle, the needle tip is pulled out of a needle sheath and pierced into the lesion along a direction perpendicular to the wall of the bronchus with a depth of 0.5-1.2 cm. Then, a 20-50 ml syringe is connected to continue negative pressure suction and aspirate the biopsy needle up and down for 3 to 5 times. And then the aspiration was stopped, the needle tip was returned to the sheath, and the puncture needle was withdrawn.

4. Putting the removed tissue on the slide, fixing it with alcohol, and immediately sending it to the pathology.

5. Controlling the slave robot to withdraw the bronchoscope and biopsy instrument from the respiratory tract of the patient.

6. Disinfecting the slave robot.

In this disclosure, after the doctor operates the master robot, the master host sends motion information to the slave robot, and the slave robot receives an instruction sent by the slave embedded controller, so as to complete an operation on a corresponding task object by a driver, such as pushing or pulling, rotating, and bending control; and the slave robot feeds back position and speed information to the slave embedded controller by the driver.

In this disclosure, the electromagnetic navigation uses an Aurora electromagnetic tracking system of NDI company, and the visual navigation uses a display lens included in the bronchoscope. Two navigation manners are cooperatively used.

In this disclosure, the doctor may install a common bronchoscope on the robot. In an operation process, the doctor may also select different biopsy instruments such as the biopsy forceps, the biopsy needle, etc., according to a requirement.

In this disclosure, after a doctor manually inserts the biopsy forceps or the biopsy needle into the biopsy channel of the bronchoscope, the biopsy instrument introduction mechanism 13 delivers the biopsy forceps or the biopsy forceps into a lesion for biopsy sampling by the friction wheel.

The present disclosure is described with reference to current preferred implementations. However, it should be understood by a person skilled in the art that the foregoing preferred implementations are merely intended to describe the present disclosure, and are not intended to limit the protection scope of the present disclosure. Any modification, equivalent replacement, or improvement made without departing from the spirit and the principle of the present disclosure shall fall within the protection scope of the present disclosure.

What is claimed is:

1. A robotic system for respiratory diagnosis and treatment, comprising:
a master control apparatus, configured to perform and control to send a control instruction;
a slave control apparatus, configured to receive the control instruction of the master control apparatus and performing an operation action; and
a navigation apparatus, configured to track and guide the slave control apparatus; and
wherein the slave control apparatus comprises a slave robot and a slave embedded controller installed in the slave robot, and the slave robot comprises a mechanical arm positioned on the slave robot, a propulsion support plate connected to the mechanical arm, a biopsy instrument introduction mechanism connected to the propulsion support plate and configured to deliver a biopsy instrument, and a bronchoscope rotation driving mechanism configured to control a bronchoscope.

2. The robotic system for respiratory diagnosis and treatment according to claim 1, wherein the slave robot further comprises a bending control mechanism connected to the bronchoscope rotation driving mechanism and configured to adjust an end catheter of the bronchoscope.

3. The robotic system for respiratory diagnosis and treatment according to claim 2, wherein the bronchoscope rotation driving mechanism comprises a rotation mechanism motor, a worm, a bearing support, a worm wheel, and a rotation support shaft seat, the rotation mechanism motor is connected to the propulsion support plate by a motor support, the bearing support is fixedly connected to the propulsion support plate, the rotation mechanism motor is connected to the bearing support by the worm, the worm engages with the worm wheel, the rotation support shaft seat is connected to the worm wheel, the rotation support shaft seat is connected to the propulsion support plate by a rotation support shaft.

4. The robotic system for respiratory diagnosis and treatment according to claim 3, wherein the bending control mechanism comprises a bending control knob, a bending control knob groove, a worm wheel rotation sliding block, a bending control worm, and a bending control motor, and the bending control motor and the worm wheel rotation sliding block are connected to the propulsion support plate, the bending control motor is connected to the bending control worm, the bending control worm engages with a worm wheel positioned inside the worm wheel rotation sliding block, and the bending control knob is received in the bending control knob groove defined in the worm wheel rotation sliding block and configured to lock the rotation support shaft.

5. The robotic system for respiratory diagnosis and treatment according to claim 4, wherein the biopsy instrument introduction mechanism comprises an introduction motor, a conveying roller, an inert wheel fixing shaft, an inert wheel, a guide rail, a spring, and a guide rail stopper, and the introduction motor is connected to a base plate; and the base plate is connected to the propulsion support plate, the conveying roller is connected to the introduction motor, the inert wheel is axially connected to the inert wheel fixing shaft, the inert wheel fixing shaft is connected to the guide rail and is connected to the guide rail stopper by the spring, and the inert wheel and the conveying roller are configured to guide the biopsy instrument to be rolled into a biopsy channel of the end catheter of the bronchoscope.

6. The robotic system for respiratory diagnosis and treatment according to claim 1, wherein the master control apparatus comprises a master computer host electrically connected to a master robot, and the master computer host comprises a motion controller configured to send the control instruction.

7. The robotic system for respiratory diagnosis and treatment according to claim 1, wherein the navigation apparatus comprises a visual display mechanism configured to display and an electromagnetic navigation end mechanism configured to guide the slave control apparatus.

8. The robotic system for respiratory diagnosis and treatment according to claim 7, wherein the visual display mechanism comprises a visual flexible laryngoscope.

9. The robotic system for respiratory diagnosis and treatment according to claim 7, wherein the electromagnetic navigation end mechanism comprises an electromagnetic sensor, the electromagnetic navigation system is installed on a tray on an operating table, and the electromagnetic navigation system comprises a magnetic field generator.

10. A robotic control method for a respiratory diagnosis and treatment, comprising the following steps:
step S1, fixing a bronchoscope to a slave robot of a slave control apparatus;
step S2, operating a master control apparatus to send a control instruction to control the slave robot to deliver a bronchoscope and a biopsy instrument to a predetermined aspiration and biopsy position in a respiratory tract of a patient;
step S3, controlling the biopsy instrument to perform an operation, and take out a lesion tissue;
step S4, putting the taken-out lesion tissue on a slide, fixing the lesion with alcohol, and immediately sending the lesion tissue to a pathological examination;
step S5: controlling the slave robot to withdraw the bronchoscope and the biopsy instrument from the respiratory tract of the patient;
step S6: disinfecting the slave robot.
